(19) United States
(12) Patent Application Publication
(10) Pub. No.: US 2016/0095736 A1
(43) Pub. Date: Apr. 7, 2016

(54) Splint

(71) Applicant: Pranesh KUMAR, Rotorua (NZ)

(72) Inventor: Pranesh KUMAR, Hamilton (NZ)

(21) Appl. No.: 14/968,792

(22) Filed: Dec. 14, 2015

Related U.S. Application Data
(63) Continuation of application No. PCT/NZ2014/000117, filed on Jun. 12, 2014.

(30) Foreign Application Priority Data
Jun. 12, 2013 (NZ) 604441

(51) Int. Cl. A61F 5/058

(52) U.S. Cl. CPC A61F 5/05866; A61F 5/0585; A61F 2005/0158

(57) ABSTRACT

A splint with at least two restraint portions is used for reducing a bone fracture in a body part that has a first surface and a second surface, where the bone fracture has produced at least a first bone fragment and a second bone fragment. The splint includes a first restraint portion configured to apply pressure directly to the first surface of the body part and a second restraint portion configured to apply pressure directly to the second surface. The first and second restraint portions thereby apply pressure to the first and second bone fragments, respectively. The orientation of the first restraint portion and the second restraint portion can be adjusted to align the first bone fragment and the second bone fragment in a desired orientation that substantially corresponds to an orientation for the first bone fragment and the second bone fragment in a non-fractured bone. A connection assembly between the first restraint portion and the second restraint portion holds the first restraint portion and the second restraint portion substantially immovably with respect to each other.

### CROSS-REFERENCE TO RELATED APPLICATIONS

This application is a continuation of parent International Patent Application No. PCT/NZ2014/000117, filed on Jun. 12, 2014, and published as International Patent Application No. WO 2014/200366, which claims priority to New Zealand Patent Application No. 604441, filed on Jun. 12, 2013. The entire disclosure of each prior application is hereby incorporated by reference in its entirety.

### TECHNICAL FIELD

The invention relates to a splint.

### BACKGROUND ART

The human arm is a complex composite of tissue and bone, and requires specific conditions to be met in order to promote or maximise healing from an injury. Bone fractures in particular may heal more effectively if the bone fragments are re-aligned to their original orientations, and supported in this position while the healing process repairs the damage to a stage where the bone no longer requires support.

The required support is typically needed over an extended time frame, often ranging from several weeks to 3 months or more.

With many fractures such as those in the wrist, a common method for keeping the bone in the correct orientation is a plaster or fibre glass cast. The advantage is that it can be easily tailored to the individual patient and effectively holds the bones in place. Disadvantages arise from the encasing and permanent geometry of the cast. For instance, in the event that two bone fragments are misaligned, the cast must be broken, and a new cast made. The cast also cannot be adjusted when the initial swelling around the fracture subsides.

Plaster or fibre glass casts also restrict movement of a joint near the fracture. However, movement at the joint can be important for effective healing. Alternatively, complete immobilisation of a joint can be an inconvenience for the patient.

These casts can also have other also problems including that it is difficult to clean the skin encased by the cast, which leads to an unhygienic build-up of sweat and dirt under the cast.

It would therefore be an advantage to have a splint which could assist with aligning two bone fragments created by a fracture to a desired orientation such as that corresponding to a non-fractured bone, and which can hold those bone fragments in that orientation.

Alternatively, it would be an advantage to have a splint which can permit at least a degree of movement of a joint near a fracture.

It would also be an advantage to have a splint that can be easily adjusted after being put on a patient and which does not require discarding of the cast.

Alternatively, It is an object of the present invention to address the problems with the prior art or to at least provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

Preferred aspects of the invention are set forth in the appended claims. Particular embodiments are described below in non-limiting terms.

According to one aspect of the present invention there is provided a splint for use in reducing a bone fracture that has produced at least a first bone fragment and a second bone fragment, wherein the splint includes a first restraint portion, a second restraint portion, wherein the restraint portions can be adjusted with respect to each other to thereby align the first bone fragment and the second bone fragment in a desired orientation that substantially corresponds to an unfractured position of the bone, and a connection assembly between the first restraint portion and second restraint portion, and wherein the connection assembly can hold the first restraint portion and the second restraint portion substantially immovably with respect to each other to thereby hold the first bone fragment and the second bone fragment in the desired orientation.

According to another aspect of the present invention, there is provided a method of reducing a fracture in a bone that has produced at least a first bone fragment and a second bone fragment, wherein the method uses a splint including a first restraint portion, a second restraint portion, and a connection assembly, the method including the steps of: (a) positioning the first restraint portion so as to be adjacent a first body part; (b) positioning the second restraint portion so as to be adjacent a second body part; (c) adjusting the first restraint portion and the second restraint portion with respect to each other so as to move the first bone fragment and the second bone fragment into a desired orientation that substantially corresponds to an unfractured position of the bone; (c) using the connection assembly to hold the first restraint portion and second restraint portion substantially immovably with respect to each other to thereby hold the first bone fragment and the second bone fragment in the desired orientation.

Throughout the present specification reference to the term “splint” should be understood as meaning a device configured to hold two body parts in a substantially immovable position with respect to each other. This is as should be understood by one skilled in the art.

An embodiment of the invention may be intended for use with people. Accordingly, reference will be made herein to use of the present inventions as such. However, this should not be seen as limiting on the scope of the present invention as it is also envisaged that an embodiment could be utilised with non-human animals such as horses.

Throughout the present specification reference to the term “body part” should be understood as meaning at least a section of a limb. The body part can be any body parts such as a human's an arm, wrist, leg, shoulder, elbow, or finger.

These body parts contain bones, which may be fractured to produce at least two bone fragments. Therefore, the splint may assist in holding the bone fragments in a desired orientation.

In an embodiment, the splint may be configured to allow at least partial movement at a joint near a fracture. For instance, the partial movement may be a range of motion for the joint which can assist in holding two bone fragments in a desired orientation.

Throughout the present specification reference to the term “restraint portion” should be understood as meaning a component that can bear against a surface of a body part.

In an embodiment, when the splint is in use, the restraint portion interacts with another component to hold two bone fragments in a desired orientation.

In an embodiment, a restraint portion may be a substantially rigid component. Therefore, the restraint portion may be able to bear against a body part to apply pressure thereto. This may assist with manipulating one or more bone fragments into a desired orientation. In addition, it may enable the invention to hold one or more bone fragments in a desired orientation.

In an embodiment a restraint portion may be substantially rigid at the time that when it is applied to a person. This excludes prior art casts such as plaster or fibre glass, which harden once the splint is fitted.

In an embodiment, the invention may include only two restraint portions.

However, this should not be seen as limiting on the scope of the present invention. It is also envisaged that the split may include three or more restraint portions. For instance, the splint may include two restraint portions which in use can be positioned on the same side of a body part. A third retrain portion could be positioned on a distal side of the body part to the first and second restrain portions. The first, second and third restraint portions interact with each other (potentially with other components) to move the bone fragments into a desired orientation and hold them therein.

In an embodiment a restraint portion may include at least one coating layer.

In embodiments, the coating layer may be a material which is soft, pliable, capable of pressure distribution, hydrophobic, breathable and/or hypoallergenic.

The coating layer may be a soft material such as a closed cell foam, polyurethane foam, plastics sheet, a breathable silicone, and/or a memory foam.

The coating layer may make the splint more comfortable to wear. Alternatively, the materials may make the splint more suitable for use such as by reducing the chances of an allergic reaction occurring, or reducing unpleasant odors developing. Accordingly, the splint may be a better solution to treat fractures and promote healing.

In addition, the coating layer could facilitate easy reuse of the splint. For instance, the coating layer could be replaceable, allowing the same splint to be used with different patients while minimising potential health issues. Accordingly, the coating layer could be useful to assist in making the splint re-useable.

Throughout the present specification reference to the term “locking mechanism” should be understood as meaning a component to prevent movement of about a joint. Thereby, the locking mechanism may assist to secure the first and second restraints substantially immovably with respect to each other.

Throughout the present specification, reference to the term “connection assembly” should be understood as meaning a component that connects at least two restraint portions to each other.

In an embodiment, a connection assembly may include a spacer element. The spacer element may be a component which in use is substantially rigid. That is, when the splint is secured to a person and in use, the substantially rigid component provides an element that resists and/or prevents movement of the restraint portions with respect to each other.

In an embodiment, the spacer element may be incompressible along its length. However, the spacer element may have a degree of flexibility. That is, the spacer element may be flexible and able to twist, flex, or bend.

The degree of flexibility may be useful to help assemble and/or position the splint. However, when in use the incompressibility of the spacer element assists in holding two restraint portions with respect to each other.

For instance, the spacer element may be shaped and/or configured to provide it with a degree of flexibility. However, when the splint is assembled, the spacer element is in a position in which it is substantially rigid.

Various embodiments of the space element are envisaged, including rigid rods, flexible lengths of material and/or incompressible lengths of material. Accordingly, the discussion herein should not be seen as limiting.

In an embodiment, the invention may include a plurality of spacer elements. These may assist in holding the restraint portions with respect to each other. In addition, they may provide flexibility for the positions in which the restraints positions can be held with respect to each other.

However, the foregoing should not be seen as limiting on the scope of the present invention.

In an embodiment, a connection assembly may include a length adjustment mechanism which enables the amount of the spacer element between the two restraint portions to be altered. Therefore, the length adjustment mechanism may facilitate the invention acting as a one size fits many or all product. In addition, the length adjustment mechanism may simplify the process of fitting the invention to a patient.

In an embodiment, the length adjustment mechanism may be a ratchet-type mechanism. For instance, ratchet teeth on the spacer element can engage corresponding parts of a restraint portion to secure the components with respect to each other. However, the teeth may be optionally overcome to enable the track and guide to move with respect to each other.

In an embodiment, the spacer element may be at least one rod. The rod may be moveably connected to one or more or the restraint portions, such as by a joint. For instance, the rod may be able to slide with respect to one or more of the restraint portions, thereby allowing the separation between the restraint portions to be adjusted.

In addition, the at least one rod may be able to twist and/or slide with respect to a restraint portion to which is it attached.

Alternatively, the length adjustment mechanism may be a clamp. The clamp is configured to receive the rod. When the clamp is disengaged, the rod can twist and/or slide with respect to the clamp. This may facilitate adjustment of the orientation and position of the rigid component with respect to a restraint portion. Thereby, the orientation and separation of two restraint portions to which the spacer element is (or could be) attached can be adjusted. Therefore, the clamp may also perform the function of a joint.

The clamp may also be moveably secured to a restraint portion such as by a track and guide and/or a joint. Therefore, the orientation of the clamp, and thereby also the spacer element with respect to the restraint portion may be adjusted. This can also enable the clamp to perform the function of a joint.

In an embodiment, the invention may include a joint.

Throughout the present specification reference to the term joint should be understood as meaning an assembly, part or component that facilitates movement between two other components. For instance, the joint may facilitate changing an angle between the spacer element and a restraint portion. Therefore, the joint can facilitate adjusting the position of the restraint portions with respect to each other.

In an embodiment a joint may be a three dimensional joint.

Reference to the term “three dimensional joint” should be understood as meaning a joint which has a range of motion in three dimensions.

A number of joints are envisaged as being within the scope of invention. For instance, a joint may be a ball and socket type joint. Alternatively, the joint may be a simple two dimensional hinge which allows rotation about an axis.

In an embodiment, the joint may be lockable. Thereby, the locking mechanism assists in holding two components with respect to each other.

In embodiments, the invention may include two or more joints, such as for example one at every junction between a spacer element and a restraint portion. However, the foregoing should not be seen as limiting on the scope of the invention and it is envisaged that a single joint may be provided.

In an embodiment, the invention may include a locking mechanism.

Throughout the present specification reference to the term “locking mechanism” should be understood as meaning a component or assembly that can hold the first and second restrain portions at a desired orientation with respect to each other. In other words, the orientation of the first and second restraint portions with respect to each other can be fixed and does not rely on body parts to hold them in a desired position.

In an embodiment, the locking mechanism may substantially or completely prevent movement about a joint.

The locking mechanism may form part of a length adjustment mechanism. Therefore, the locking mechanism may allow or facilitate the separation between two restraint portions to be adjusted. This may be useful to facilitate a one-sized fits many or all splint.

The locking mechanism may assist in moving bone fragments into a desired orientation, and holding them therein. This may be useful as it can assist with fracture reduction which can promote healing of a fracture.

In an embodiment, the locking mechanism releasably secures the first and second restraint portions with respect to each other. Accordingly, the locking mechanism enables the position of two restraint portions with respect to each other to be adjusted after the splint has been put on a person, while still facilitating that the restraints can be held with respect to each other substantially immovably when desired. Once the position has been adjusted, the locking mechanism can be engaged to secure the restraint portions with respect to each other.

It should be understood that the term “releasably” excludes prior art casts such as those made from plaster or fibre glass. In these, the cast must be broken using a cutting device such as a saw. Therefore, the orientation of the cast cannot be adjusted after it has been put on a person.

The locking mechanism may take various forms and therefore these are envisaged as being within the scope of the invention. For instance, the locking mechanism may be a clamp, hinge, ratchet arrangement or other combination of components. All of these embodiments can be placed in a position in which they fixedly and releasably restrict or prevent movement of the first restraint portion and the second restraint portion with respect to each other.

In an embodiment, the desired orientation substantially or completely corresponds to an orientation of two bone fragments in a normal un-fractured (non-fractured bone). Therefore, the invention may facilitate the bone fragments being brought into an orientation in which they align and which substantially corresponds to the non-fractured bone. This reduces the fracture.

Reference throughout the present specification to the term “desired orientation” should be understood as meaning a position for at least two bone fragments with respect to each other.

The desired orientation may reduced a fracture, and therefore promote healing of the fractured bone. In-use, an embodiment of the restraint portions may be used to move at least two bone fragments into the desired orientation. The locking mechanism can subsequently hold the restraint portions with respect to each other substantially immovably. The locking mechanism operation is not reliant on pressure or contact with a body part. Accordingly, there is a greater variability with the bone fractures and/or people that could be treated using the invention.

The invention has been described with reference to what is presently considered to be the most practical and preferred embodiments. However, it should be understood that the invention is not limited to only the disclosed embodiments. Rather, the invention can include various modifications and equivalent arrangements which are within the spirit and scope of the invention. The embodiments of the invention described herein may be implemented in conjunction with other described embodiments, e.g., aspects of one embodiment may be combined with aspects of another embodiment to realise yet other embodiments. Further, each independent feature or component of any given assembly may constitute an additional embodiment.

### BRIEF DESCRIPTION OF THE DRAWINGS

Further aspects of the present invention will become apparent from the ensuing description which is given by way of example only and with reference to the accompanying drawings in which:

FIG. 1 is a side view of a patient's wrist in a normal (non-fractured) state;

FIG. 2 is a view of FIG. 1 showing a representative wrist fracture with which an embodiment of the invention may be used and the orientation of bone fragments;

FIG. 3 is a first perspective view of an embodiment of a wrist splint according to an embodiment of the present invention

FIG. 4 is a side view of FIG. 3;

FIG. 5 is a bottom view of FIG. 3;

FIG. 6 is a top view of FIG. 3;

FIG. 7 is a first end view of FIG. 3;

FIG. 8 is a second perspective view of FIG. 3;

FIG. 9 is a side view of a wrist splint according to an embodiment of the present invention in-use;

FIG. 10 is an alternate wrist splint according to an embodiment of the present invention on a patient's arm;

FIG. 11 is a side view of FIG. 10;

FIG. 12 is a view of the wrist splint of FIGS. 10 and 11;

FIG. 13 is a perspective view of a first restrain portion and a second restraint portion and a second restraint portion of the wrist splint of FIGS. 10-12;

FIG. 14 shows perspective views of rods forming part of a connection assembly according to the wrist splint of FIGS. 10-13;

FIG. 15 is a perspective view of a locking mechanism of the embodiment of FIGS. 10-13;

FIG. 16 is a first view of an ankle splint according to an embodiment of the present invention;

FIG. 17 is a perspective view of FIG. 16;

FIG. 18 is a perspective view of the first restraint portion and second restraint portion of the embodiment shown in FIGS. 16 and 17;

FIG. 19 is a view of connection assembly according to the embodiment shown in FIGS. 16-18;

FIG. 20 is an embodiment of a locking mechanism according to an embodiment of the invention shown in FIGS. 16-19;

FIG. 21 is a perspective view of a hinge according to an embodiment of the present invention;

FIG. 22 is a perspective view showing the embodiment of FIGS. 16-21 in-use;

FIG. 23 is a first perspective view of an exploded assembly of a splint according to FIG. 3;

FIG. 24 is an alternate perspective view of FIG. 23;

FIG. 25 is side view of FIG. 23;

FIG. 26 is a first perspective view of a finger splint according to an embodiment of the invention;

FIG. 27 is an alternative perspective view of FIG. 26; and

FIG. 28 is a side cross sectional view showing the splint of FIGS. 26 and 27 in use.

### BEST MODES FOR CARRYING OUT THE INVENTION

### Wrist Fractures

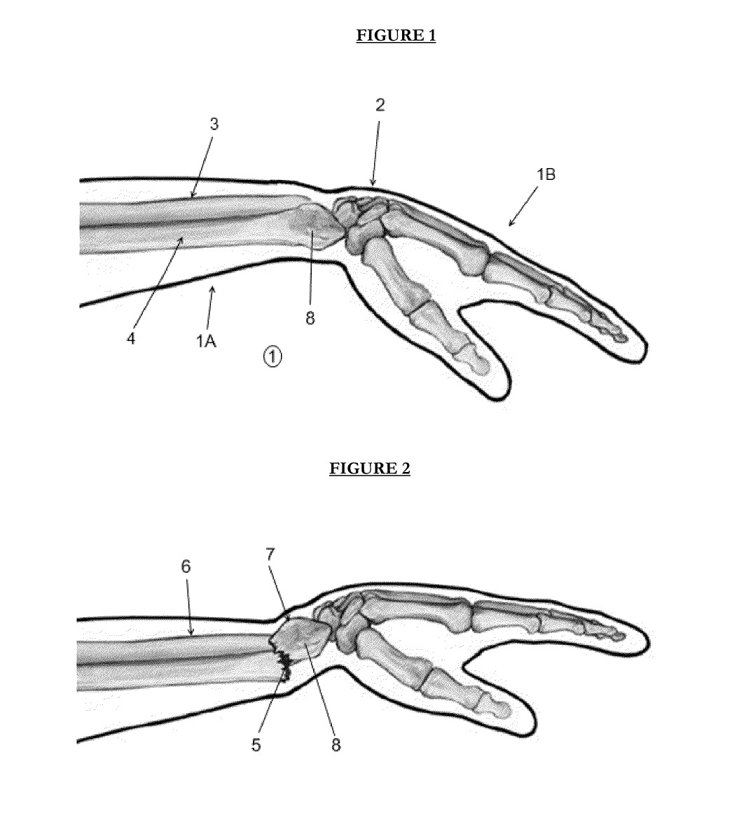

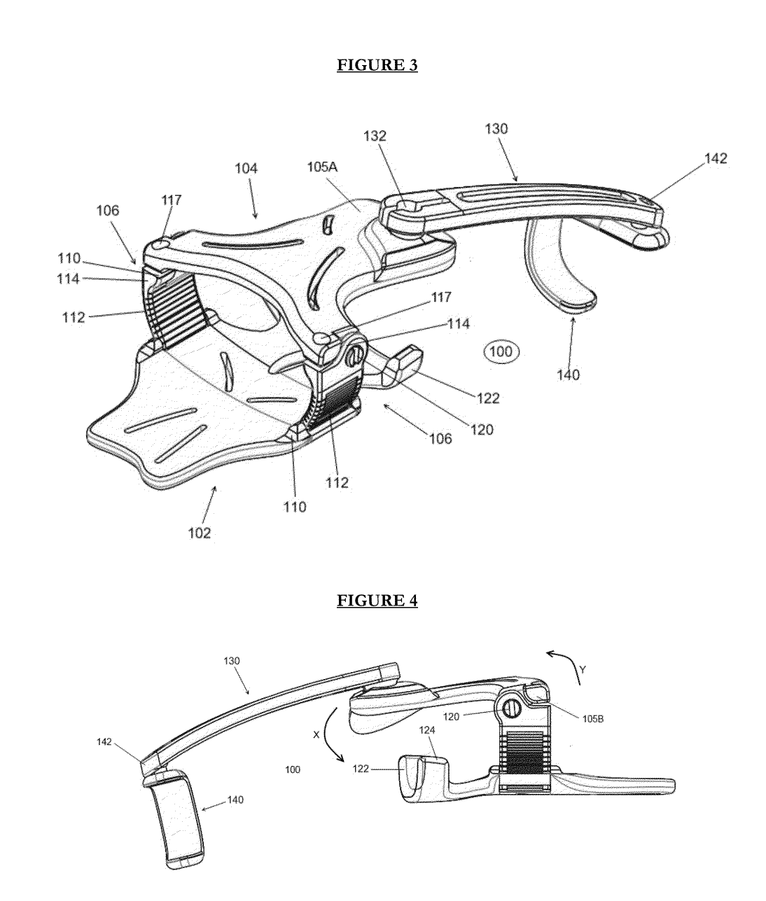

Referring first to FIGS. 1 and 2 which show a side cross sectional view of a person's forearm (1A), hand (1B) and wrist joint (2) generally as would be obtained using an X-ray. The wrist joint (2) provides motion between the bones of the forearm (1A), being the ulna (3) and radius (4), and the carpal (not indicated) in the hand (1B).

A common wrist fracture is shown in FIG. 2, where a crack or break (5) occurs in the radius (4) which creates a first bone fragment (6) and a second bone fragment (7).

### Wrist Splint

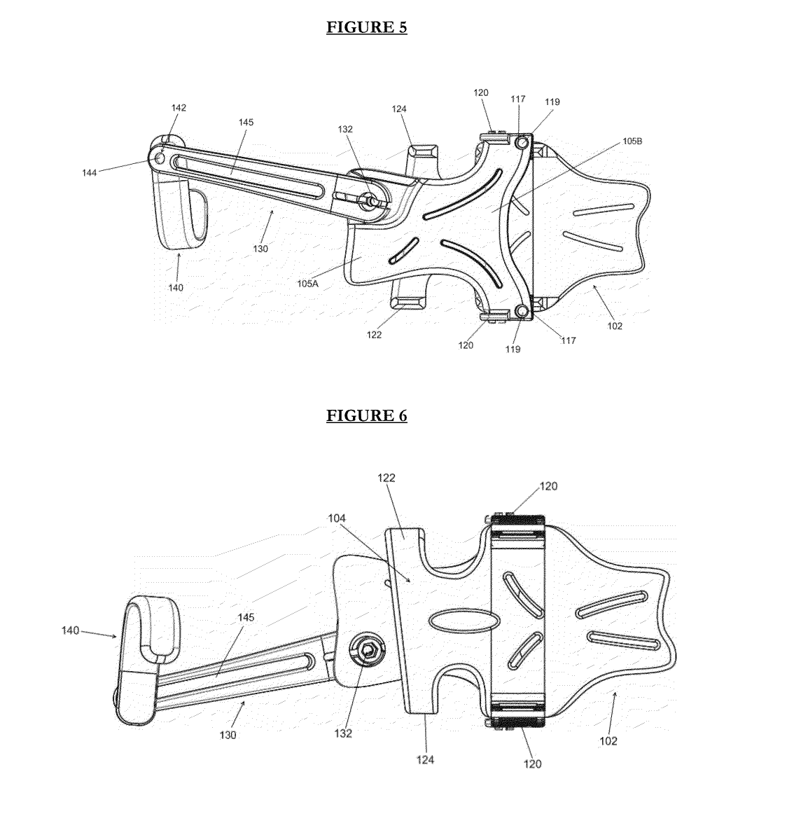

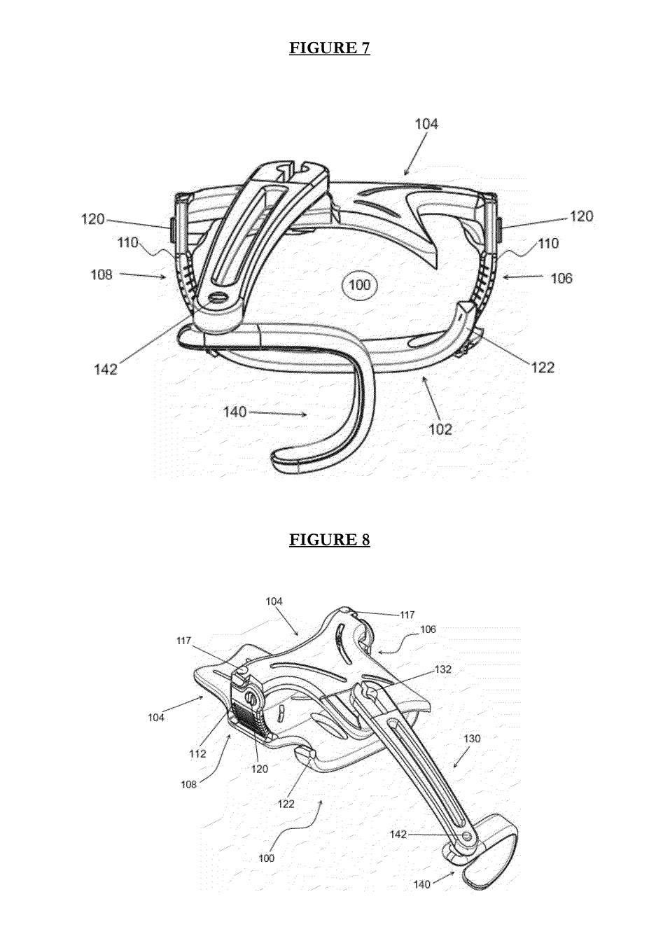

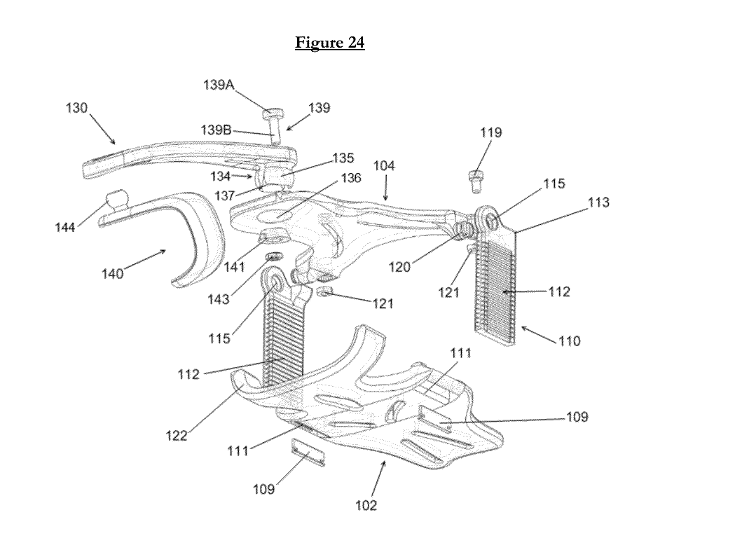

Referring now to FIGS. 1 to 9 and 23 to 25, which show an embodiment of a wrist splint (100). The wrist splint (100) may be provided as a kitset of separate parts or fully assembled and ready to use.

The components of the wrist splint (100) may be made from different materials and/or techniques. For instance, they may be made by injection moulding of plastics materials, 3 dimensional printing techniques using plastic and/or metals, CNC machining of metal components, or shaping and bending sheets of metal and/or plastics.

The wrist splint (100) includes a first restraint portion (102) and a second restraint portion (104).

The first restraint portion (102) provides a dorsal pad which in use can abut and lay against the dorsal surface (not indicated) of a person's forearm (1A). The second restraint (104) provides a volar pad which in use can abut and lay against the volar surface (not indicated) of a person's forearm (1A).

The restraint portions (102, 104) are connected together by a first connection assembly (106) and a second connection assembly (108).

The connection assemblies (106, 108) are identical to each other and therefore only connection assembly (106) will be described herein. However, identical reference numerals in the Figures are used to refer to identical components.

The connection assembly (106) includes a spacer element (110). The spacer element (110) has a plurality of teeth (112) formed integrally to an outer surface. The spacer element (110) is resilient and able to flex from a substantially linear position into a curved position. However, the spacer element (110) is substantially incompressible along its length.

The first restraint portion (102) has a channel (111) which is configured to receive the spacer element (110). In use, the teeth (112) engage the edges of the channel (111) to releasably secure the spacer element (110) with respect to the first restraint portion. A latch (109) as pivotally mounted to first restraint portion (102) adjacent to the channel (111). In-use, the latch (109) can be moved to a locking position in which it secures the spacer element (110). The latch (109) is held in the locking position by engaging with the protrusions (not shown) on the first restraint component in a snap-fit engagement.

In combination, the spacer element (110), channel (111), and ratchet teeth (112) provide a length adjustment mechanism and locking mechanism. This facilitates adjusting the amount of the spacer element (110) between the first and second restraint portions (102, 104) and also releasably holding the first and second restraint portions with respect to each other.

The spacer element (110) has a shoulder (113).

A hinge is provided in the shoulder between the track and the second restraint portion (104). The hinge is formed by a screw (120) formed integrally to the second restraint portion (104). The screw (120) is configured to extend through an aperture (115) in the shoulder (113).

In use, the screw (120) can be rotated. In doing so, the screw (120) causes the second restraint portion (104) to rotate about an axis defined by the length of the screw (120). This causes an end (105A) of the second restraint (104) portion to move in the direction shown by arrow (X) in FIG. 4. In addition, end (105A) of the second restraint portion (104) moves in the direction shown by arrow (Y) in FIG. 4. Accordingly, the separation between end (105A) and the shoulder (113) is increased.

A locking mechanism is provided to prevent rotation about the screw (120). In the embodiment shown, the locking mechanism is formed by a pair of channels in the second restraint portion (104). Fasteners (119) can therefore be inserted through channels (117) and engage corresponding nuts (113). The fasteners (119) can be rotated so that nuts (121) bear against shoulder (113). Therefore, the fasteners prevent rotation of the second restraint portion (104) in directions counter to arrows (X, Y) in FIG. 4.

The first restraint portion (102) is shaped and configured to define a cradle which corresponds to the dorsal surface (12) of a person's forearm (1A).

A pair of spaced about protrusions (122, 124) curve upwardly from the first restraint portion (102). In use, the protrusions sit on either side of the person's forearm (1A) and assist in restricting or preventing lateral movement of the forearm (1A) with respect to the splint (100).

A third restraint portion (130) is moveably mounted to the second restraint portion (104) by a joint (132). The joint (132) is a ball and socket type joint which provides a range of motion for the second restraint portion (104) and third restraint portion (130) with respect to each other.

The joint components (132) are shown in more detail in FIGS. 23 to 25, and generally include a ball (134) formed integrally to the third restraint portion, and a socket (136) formed integrally to the second restraint portion (104). The ball (134) has an outer clamping surface (135) and a cavity (137). A spacer (138) has a shape corresponding to the cavity (137).

A screw (139) has a screw head (139A) and a threaded body (139B). The screw (139) can be positioned to extend through apertures in the ball (134) and socket (126). In use, the threaded body (139B) engages a corresponding screw thread in a nut (143). The nut (143) forces a spacer into the cavity in ball (134). This forces the clamping surface (135) outwards to engage the walls that define the socket. Therefore, the screw (139), nut (143) and spacer (141) clamp the ball (134) and socket (136) together to hold them substantially immovably with respect to each other. Accordingly, the hinge (132) includes a locking mechanism to prevent movement of the second and third restraint portions with respect to each other.

A fourth restraint portion (140) is moveably secured to the third restraint portion (130). In the embodiment shown, a ball (144) is integrally formed to the fourth restraint portion (140). A channel (145) is formed in the third restraint portion. The channel (145) is configured to receive the ball (144) in a press fit manner. The channel (145) is shaped to allow the ball (144) to slide therein and to rotate. However, the channel (145) provides resistance to the ball being withdrawn.

The fourth restraint portion (140) has a generally U or C shaped cross section as can be best seen in FIGS. 3, 4, and 7.

The first restraint portion (102) is moved so that the cradle receives the volar surface of the person's forearm.

The second restrain portion is positioned so that the spacer elements (112) align with the channels (111). This may require pressure to be applied to the spacer elements (110) to cause these to flex. The second restraint portion (104) is moved towards the dorsal surface of the person's forearm (1A). The spacer elements (110) therefore pass through the channels (111). The teeth (112) and channels engage each other and act as a ratchet that secures the track (112) with respect to the channels (111). This provides resistance to unintended movement of the first and second components (102, 104) with respect to each other.

To adjust the separation between the first restraint portion (102) and the second restraint portion (104), the spacer elements (110) may be flexed to cause the teeth (112) to disengage the edges of the channels (111). Therefore, the spacer elements (110) can slide through the channels (111).

The orientation of the second restraint portion (104) with respect to the first restraint portion can be adjusted using screws (120). This facilitates the splint (100) applying directional pressure to the first and/or second bone fragments to move these into a desired orientation substantially corresponding to the orientation of a non-fractured bone. To do so, the screws (120) are rotated. This causes the second restraint portion (104) to pivot about an axis defined by the screws (120). Therefore end (105A) moves in the direction shown by arrow (X) in FIG. 4, while end (105B) moves in the direction shown by arrow (Y) in FIG. 4.

The latch (109) is moved to a locking position to engage the first restraint portion (102) in a snap-fit manner.

The second restraint portion (104) is used to move the second bone fragment (7) into an orientation with respect to the first bone fragment that substantially or completely corresponds to the orientation in a non-fractured bone, for instance as is shown in FIG. 1. This is achieved by rotating screws (120) to move the second restraint portion (104) pivotally.

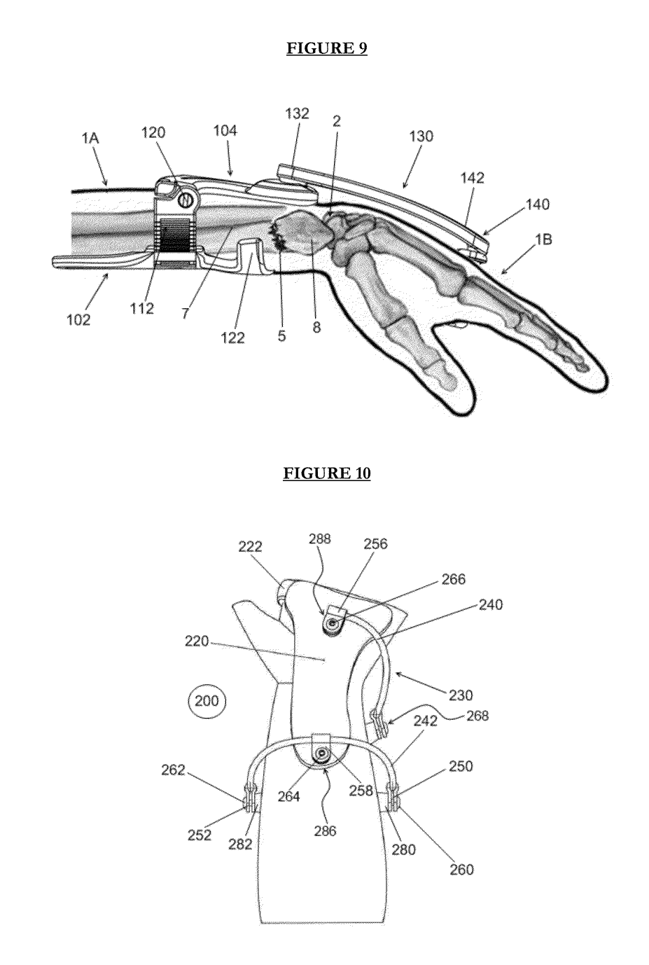

Rotation of second restraint portion (104) continues until the bone fragments (6, 8) are in the desired orientation. This position is best shown in FIG. 9. As can be seen, the second restraint portion (104) is at an angle to (inclined with respect to) the length of the first restraint portion (102). In other words, if the person were to hold their forearm horizontal then the first restraint portion (102) would be substantially horizontal while the second restraint portion (104) would be angled downwards.

The locking mechanism to secure second restraint portion (104) with respect to the first restraint portion (102) is engaged. This is by rotating screws (119) to move nuts (121) to bear against ledge (113).

The third restraint portion (130) is moved about hinge (132) so as to be substantially aligned above the person's forearm (1A) and hand (1B). In doing so, the fourth restraint portion (140) is brought into proximity to a side edge of the person's hand (1B).

Screw head (139A) is engaged to rotate screw to engage the corresponding screw thread so that spacer forces clamping surfaces (135) outwards to engage the socket walls (136). Therefore, the hinge (132) is locked to prevent the third restraint portion (130) moving with respect to the second restraint portion (104). The hinge (142) is not locked. Therefore, the fourth restraint portion (140) can move with respect to the third restraint portion (130). This may assist in providing a comfortable splint (100) as the position of the fourth restraint can be easily adjusted by the patient.

The patient can move their hand (1B) laterally away from the fourth restraint portion (140). This may facilitate movement around the wrist (2) which could promote healing of the break (5). In addition, the person may be able to perform tasks which may not have been possible with prior art splints. Therefore, comfort and performing normal tasks may be facilitated through use of the splint (100). However, the fourth restraint portion provides a restrictor, to limit the range of motion at the joint to that which will not force the bone fragments out of alignment.

### Alternate Wrist Splint

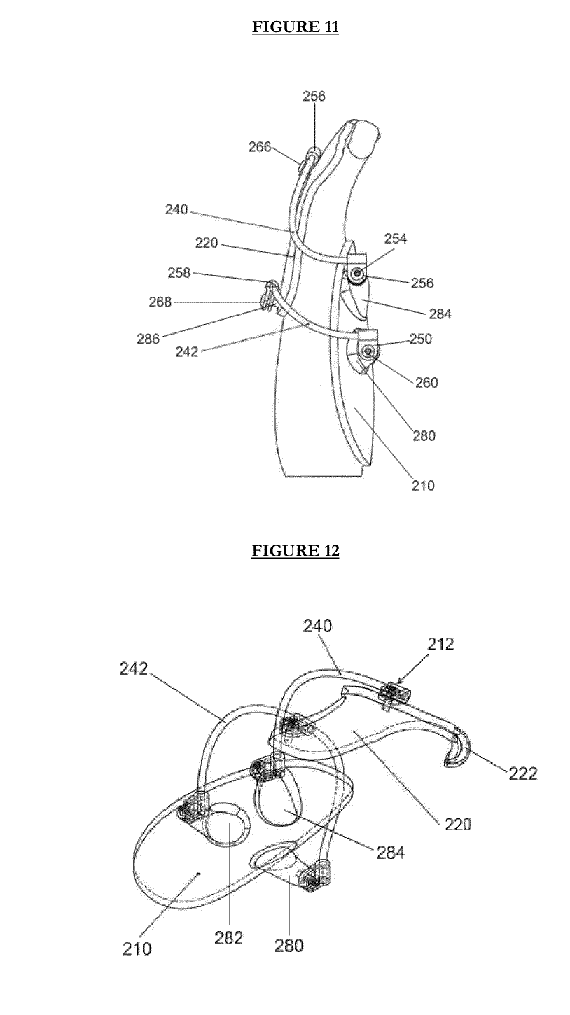

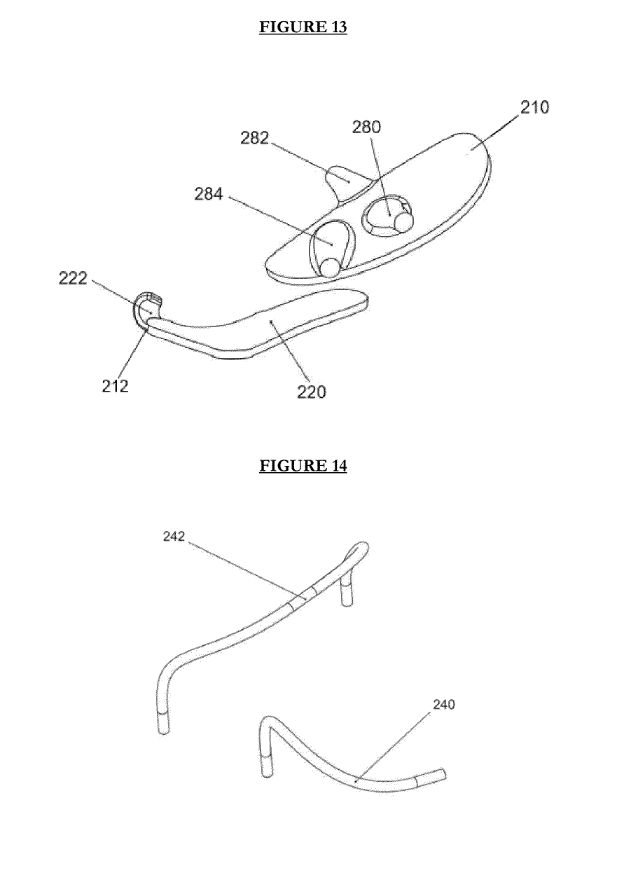

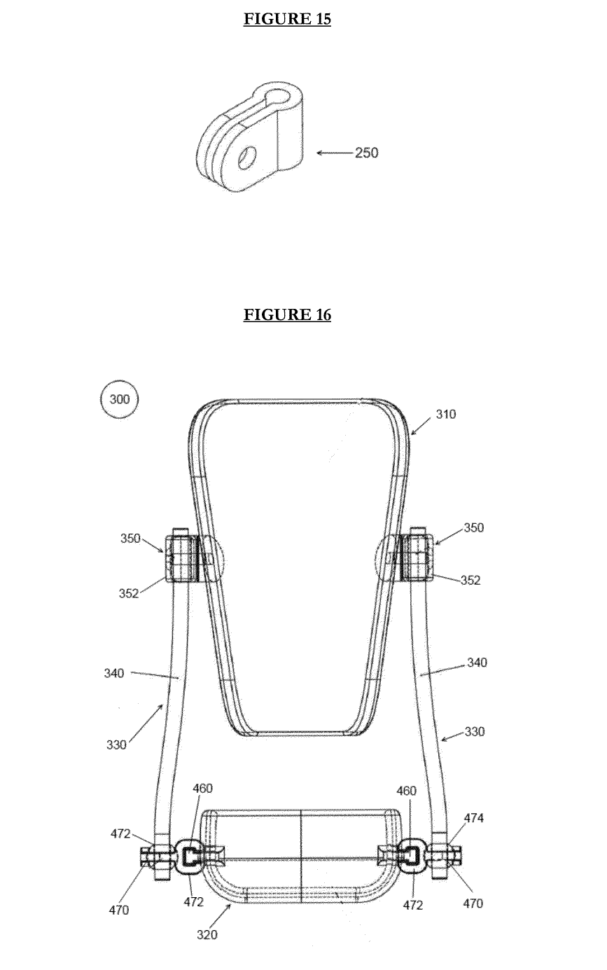

Referring now to FIGS. 10 to 15 which show an embodiment of a wrist splint (200). The wrist splint includes a first restraint portion (210), a second restraint portion (220). The wrist splint (200) can be used to treat a wrist fracture such as that discussed with reference to FIGS. 1 and 2.

The first restraint portion (210) provides a dorsal pad that is configured to in use lay against the dorsal surface of a patient's forearm. The second restraint (220) provides a volar pad that is configured to in use lay against the volar surface of a patient's forearm.

A connection assembly (230) is provided to connect the first restraint portion (210) and second restraint portion (220) to each other. In the embodiment of FIGS. 10 to 15, the connection assembly (230) is provided in the form of a rods (240, 242), clamps (250, 252, 254, 256, 258) and threaded screws (260, 262, 264, 266, 268).

The first restraint portion (210) has threaded ports (280, 282, 284) corresponding to the threaded screws (260, 262, 264). The second restraint portion (220) has threaded ports (286, 288) corresponding to threaded screws (266, 268).

The second restraint portion (220) has a C shaped restrictor (222) which extends from a side edge (212) thereof. In use, the restrictor (222) abuts a side edge of a patient's hand to prevent lateral movement. However, the patient's hand is still able to move to a small degree laterally away from the restrictor (222).

The size of the first restraint portion (210) and the second restraint portion (220) may vary between design iterations or to accommodate arms of different sizes of patient.

In one embodiment, the approximate dimensions are as follows: length will range from 60-200 mm and width at the widest point 30-100 mm.

To assemble the splint (200) the first restraint portion (210) is positioned to abut and lie against the volar surface of a patient's forearm. The second restraint portion (220) is positioned to abut and lay against the dorsal surface of the patient's forearm. The screws (260, 262, 264, 266, 268) are loosened. Rod (242) is inserted through clamps (250, 252, 256).

The orientation of second restraint portion (220) with respect to the first restraint portion (210) is adjusted by tilting the second restraint portion (220). This causes an end of the second restraint portion (220) to press against the volar surface of the patient's arm. The rod (240) acts as an axle about which the second restraint portion (220) rotates. Thereby, the second restraint portion (220) is able to apply directional pressure to bone fragments within the patient's forearm. As a result, the splint may be used to move the bone fragments into a desired orientation such as that corresponding to a non-fractured bone.

The screws (266, 268) are inserted through clamps (256, 258) and into threaded ports (282, 284). Engagement of the respective screw threads closes clamps (256, 258) and thereby secures the rod (240) with respect to the second restraint portion (220).

Interaction of the rods (240, 242) secures the first restraint portion (210) and second restraint portion (220) with respect to each other. The orientation of the restraint portions (210, 220) relative to each other is not dependent on the surfaces of the patient's forearm against which they bear. Rather, the orientation of the second restraint portion (220) can be selected so as to align the bone fragments to substantially correspond to a non-fractured bone, and the restraint portions are held in the desired position by the connection assembly.

### Ankle Splint

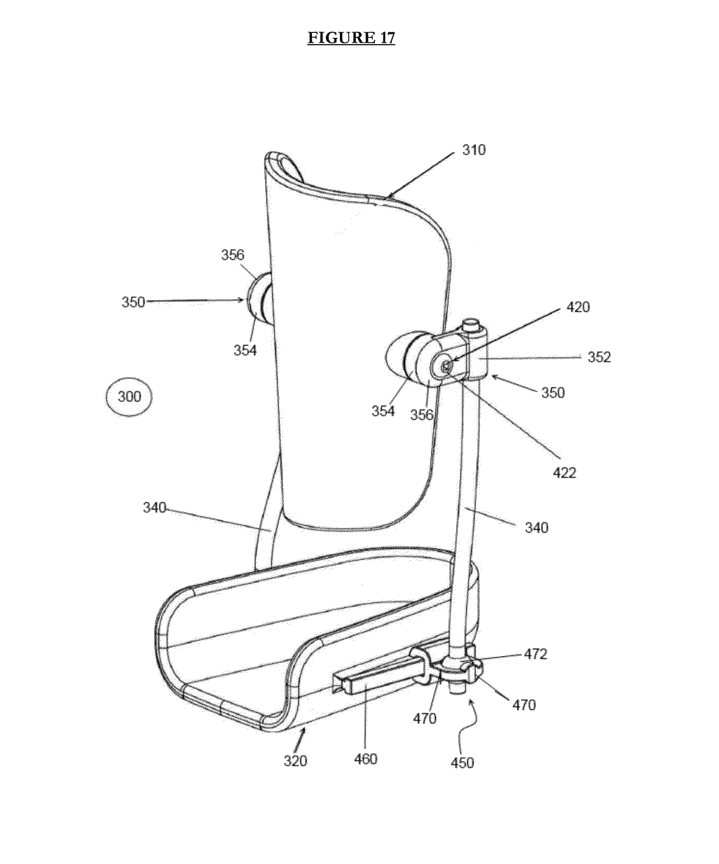

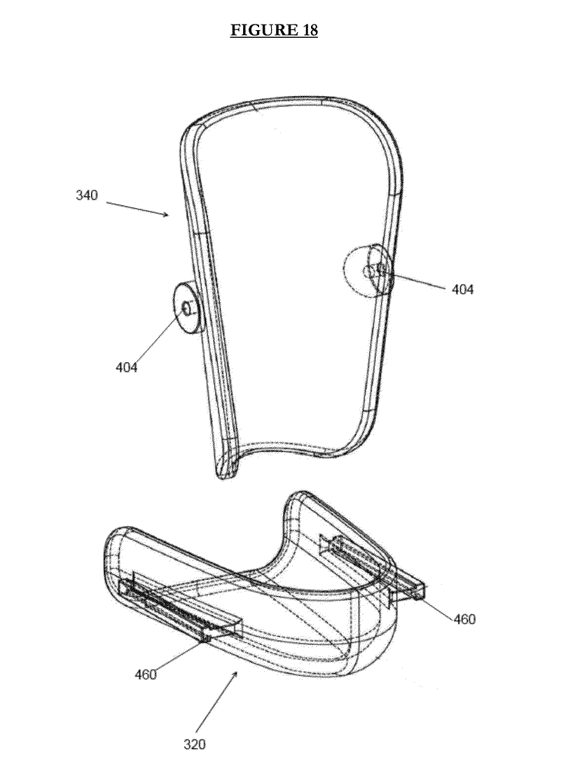

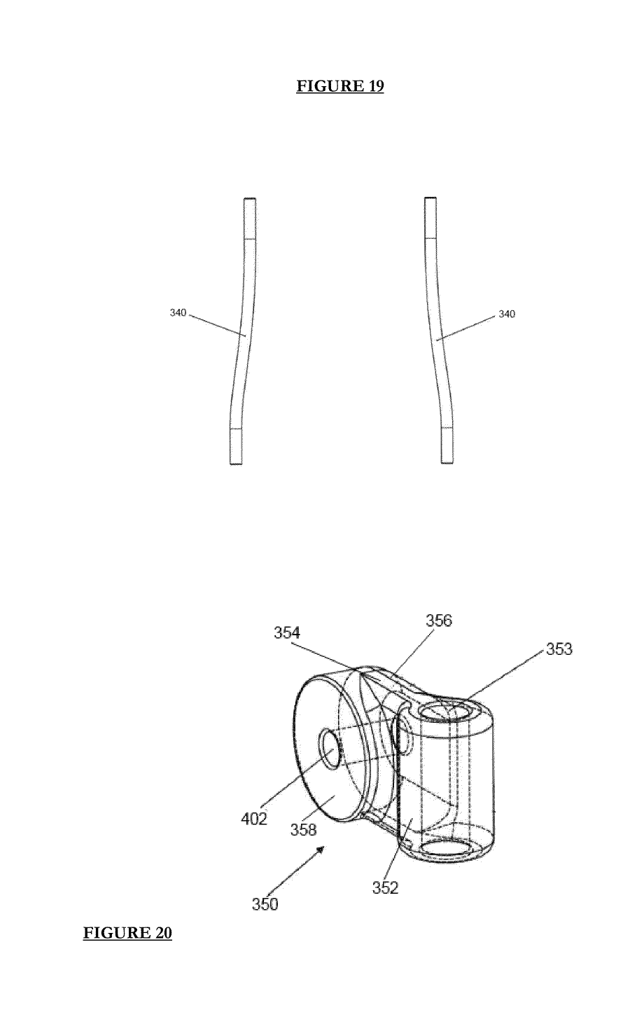

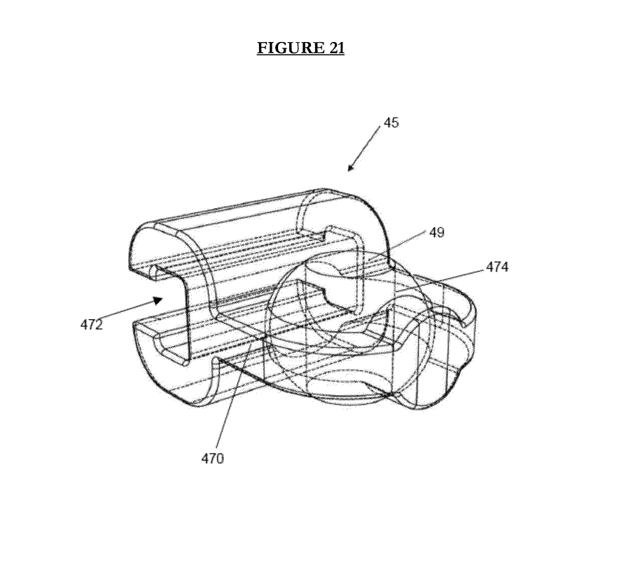

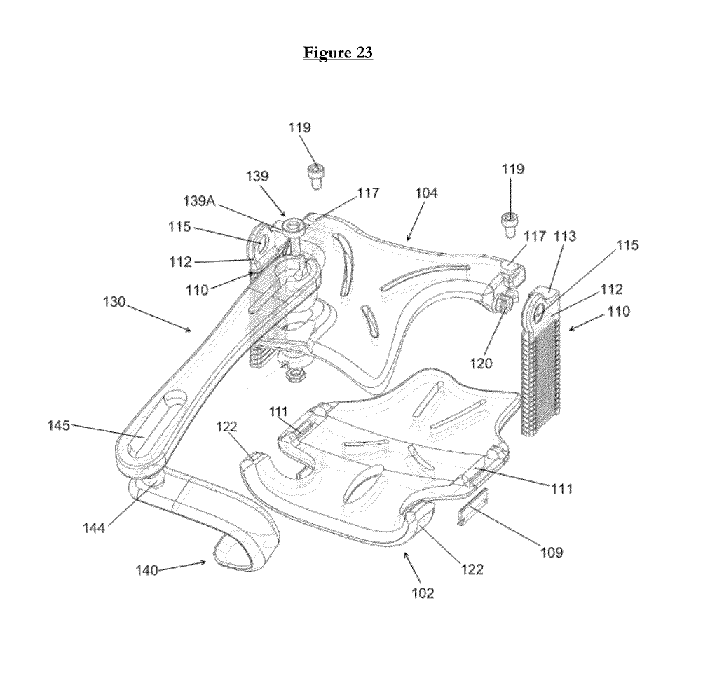

Referring now to FIGS. 16 to 22 which show an embodiment of the invention in the form of an ankle splint (300).

The ankle splint (300) includes a first restraint portion (310) and a second restraint portion (320).

The first restraint portion (310) is generally curved and shaped to wrap around a person's shin (370), being the front surface of the person's leg (371).

The second restraint portion (320) is generally shaped in the form of a cup having one open side. The second restraint portion (320) is sized and shaped so as to be able to receive the heel (not visible) of a person's foot (372).

The first restraint portion (310) and the second restraint portion (320) are rigid and formed from moulded plastic materials. In addition, the first restraint portion (310) and the second restraint portion (320) may include a coating of a cushioning material such as closed cell polyurethane foam, breathable silicon or other material having properties such as being soft, pliable, capable of pressure distribution, hydrophobic, breathable and/or hypoallergenic.

The ankle splint (300) includes two connection assemblies, each indicated generally as (330). The connection assemblies (330) are identical to each other and therefore only one of the connection assemblies (330) will be described herein.

The connection assemblies (330) include a spacer element in the form of a rigid rod (340) made from stainless steel or other rigid materials.

Each rod (340) is connected to the first restraint portion (310) by a joint that is indicated generally as (350).

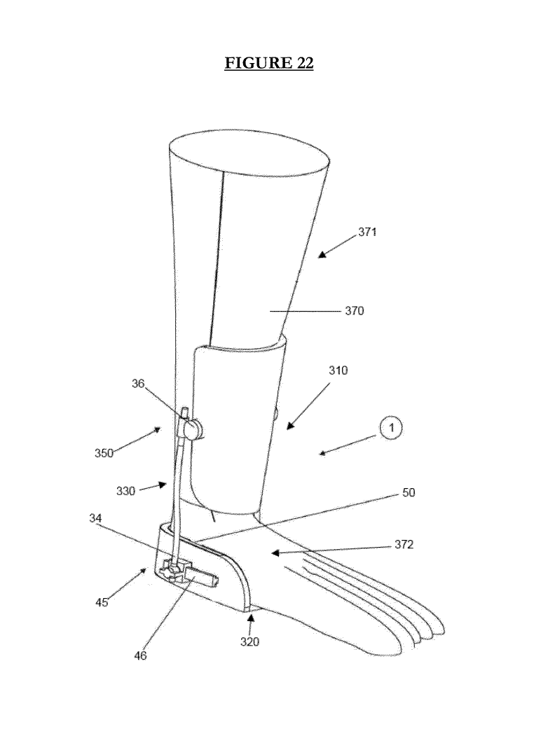

The joint (350) is shown in more detail in FIG. 20. The joint (350) includes a body (352) having a channel (353) that is configured to slidably receive rod (340). Flanges (354, 356) extend away from the body (352). Flange (354) includes a camming surface (358). Flange (356) overlaps camming surface (358) so that the two lie adjacent to each other.

The joint (350) is releasably secured to the first restraint portion (310) by a screw (420) that extends through an aperture (402) in the flanges (354, 356) and into a corresponding aperture (404) in the first restraint portion (310). The first restraint portion (310) has a camming surface (406) that is complementary to camming surface (358).

The camming surfaces (358) and (406) are shaped so that when positioned adjacent to each other the apertures (400, 402) align. The camming surfaces define a range of motion for the body (352) with respect to the first restraint portion (310).

The screw (420) has a head (422) and threaded shaft (not shown in the Figures). The head (422) is shaped and configured to be engaged by a tool such as an allen key (not shown in the Figures) as should be known to one skilled in the art.

The tool (not shown) can be used to rotate screw (420) and cause threaded shaft (not shown) to engage a corresponding thread formed inside aperture (404). The engagement of the threaded shaft (not shown) with thread (not shown) inside aperture (404) secures body (352) with respect to the first restraint portion (310).

The engagement of threaded shaft (not shown) with the thread (not shown) in the aperture (404) clamps flanges (354, 356) together. This causes sleeve the channel (353) to tighten around the rod (340) disposed therein to thereby the clamp rod (340) to the body (352). Accordingly, the rod (340) is secured with respect to the first restraint portion (310).

The connection assembly (330) includes a second joint indicated generally by (450) between rod (340) and the second restraint portion (320).

The second joint (450) includes a rail (460) secured to a side edge of the second restraint portion (320). The rail generally has a T-shaped cross section.

The second joint (450) includes a housing (470) having a channel (472). The channel (472) generally has a C-shaped or U-shaped cross section. The cross section of the channel (472) corresponds to the shape of rail (460). Accordingly, the channel (472) and therefore the housing (470) can slide along the length of the rail. However, the rail (460) and channel (472) interlock to restrict or prevent laterally (sideways) movement of the housing with respect to the rail (460).

A sleeve (474) is moveably mounted in the housing (470). The sleeve (474) is shaped to receive the rod (340) in a sliding manner. The outer walls of sleeve (474) generally have a ball shape. Interaction of the sleeve (474) and the housing (470) provides a ball and socket type joint.

The second joint (450) includes a locking mechanism. The locking mechanism can be moved between an engagement position and a release position. In the engagement position the locking mechanism secures the rod (340) substantially immovably within sleeve (474), and the housing (470) with respect to the rail (460). In the disengaged position, the rod (340) can slide and rotate with respect to the sleeve (474). Accordingly a length adjustment mechanism and locking mechanism are provided.

When the locking mechanism is in the release position the channel (472) can slide along the length of rail (460). In addition, the rod (340) can slide through sleeve (474).

In use, the locking mechanisms are released so as to enable rods (340) to move freely with respect to the joint (350, 450). In addition, the body (352) and the housing (470) are able to move freely with respect to the first and second restraint portions (310, 320). In relation to the body (352) this is able to pivot about the threaded shaft (not shown) of screw (420). In effect, the threaded shaft acts as an axle to allow the body (350) to move with a pivoting motion.

Housing (470) this is able to slide along the length of rail (460). In addition, rod (340) can slide freely through the sleeves (370) and (474).

### Ankle Splint In-Use

The locking mechanisms are disengaged to enable the position of the first and second restraint portions (310, 320) with respect to each other to be adjusted. The splint (300) is then positioned to insert the person's heel (not shown) into the second restraint portion (320). The second restraint portion (320) cups the person's heel.

The first restraint portion (310) is moved to lie adjacent to the person's shin (370). The rods (340) may be adjusted as necessary.

A tool (not shown) is used to engage fastener head (422) so as to rotate fastener (420). This causes threaded shaft (not shown) to engage with the threaded aperture (not shown) in the first restraint portion (310). Engagement of threaded shaft (not shown) and threaded aperture (not shown) clamps flanges (354, 356) together. Clamping of flanges (354, 356) causes sleeve (474) to tighten around rod (340). This secures the housing (470) with respect to the first restraint portion (310). In addition rod (340) is not able to move with respect to slide or rotate within sleeve (474). Accordingly, the rod (340) is locked in position with respect to the body (352) and therefore first restraint portion (310).

The locking mechanism is engaged to secure second hinge housing (470) with respect to second restraint portion (320).

In addition, the rod (340) is clamped within sleeve (474). Accordingly, a locking mechanism is provided that prevents the rod (340) sliding or rotating with respect to sleeve (474).

The first and second restraint portions each abut a surface of a body part. The orientation of the first and second restraint portions (310, 320) is such that they are on distal surfaces of the person's body parts. Accordingly, the first and second restraint portions (310, 320) prevent movement of the body parts with respect to each other.

The second restraint portion (320) does not restrict movement of the person's foot (372) about the ankle e.g a person can still move their foot (372) to raise their toes. This may beneficial to facilitate normal movement and to reduce the fracture within the ankle.

### Meta-Carpal Splint

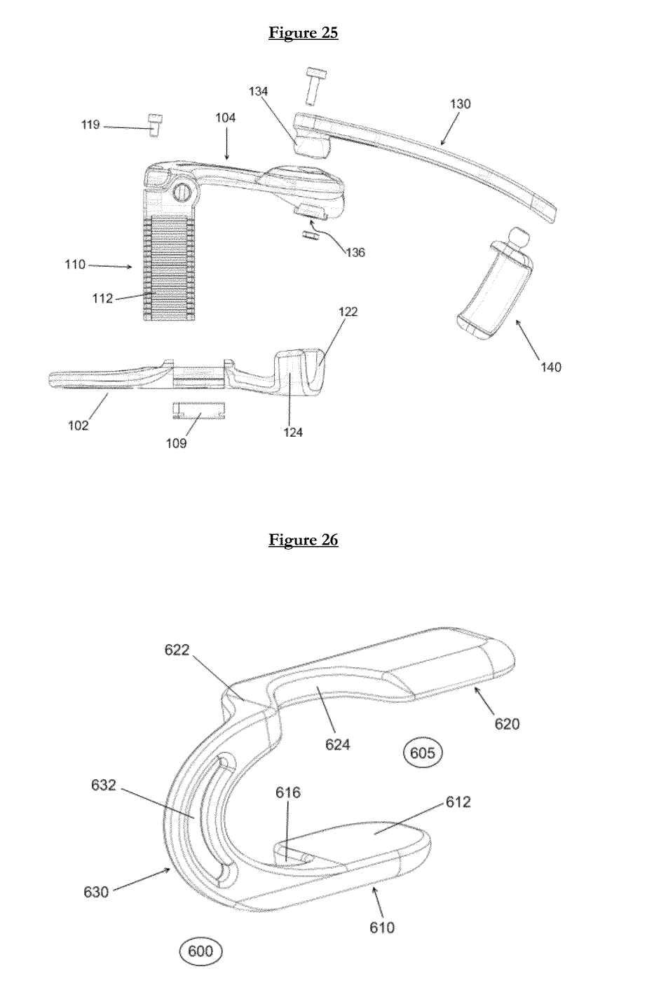

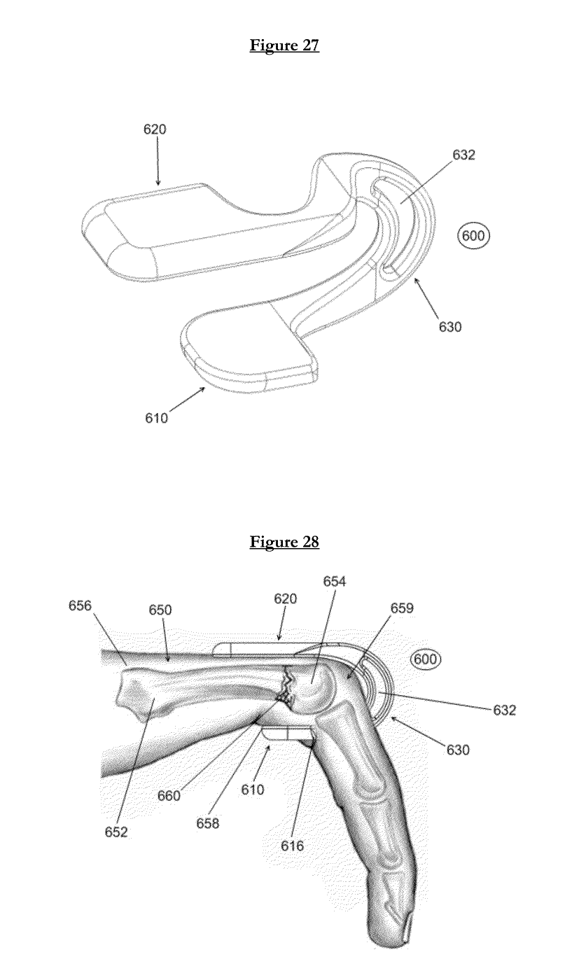

Referring now to FIGS. 26, 28 which show an embodiment of a finger splint (600). The splint (600) may be used to reduce a fracture in a meta-carpal of a person's finger

The finger splint (600) is formed as an integral, one piece unit via moulding of plastics or similar materials. In addition, the splint (600) may be provided in a range of sizes, each of which fits a different size finger.

The finger splint (600) includes a first restraint portion (610) and a second restraint portion (620).

The first restraint portion (610) and the second restraint portion (620) are connected via a connection assembly in the form of a hinge (630). The hinge (630) is resilient allowing pressure applied to the splint (600) to change the separation and orientation of the first and second restraint portions (610, 620) with respect to each other. However, the hinge (630) provides an urging force to bias the first and second restraint portions (610, 620) back towards a default position in which the hinge (630) is not deformed.

The resiliency of the hinge (630) also enables it to function as a locking mechanism, as it provides resistance to movement of the first and second restraint portions (610, 620) with respect to each other in use.

The hinge (630) may include a cut out channel (632). The cut out channel (632) may reduce the amount of material required to form the finger splint (600). In addition, the cut out channel (632) may assist in providing the hinge (630) with the required flexibility and resilience.

The finger splint (600) has a generally C-shaped cross-section. In general, the finger splint (600) has a jaw (605) defined by the first and second restraint portions (610, 620) and the hinge (630). In-use, the jaw (605) receives a person's knuckle (659).

The first restraint portion (610) has a contact surface (612). In-use, the contact surface (612) lays against an underside (658) of a person's finger. The hinge (630) is connected to a side edge (612) of the first restraint portion (610). Therefore, in-use a section (616) of the first restraint portion (610) is positioned underneath the person's knuckle. This is shown by FIG. 28. This enables the contact surface (612) to sit underneath a first bone fragment (652) and a second bone fragment (654) in a person's finger.

An elbow (622) extends away from the hinge (630). A curved section (624) extends from the elbow (622) and transitions into the second restraint portion (620). The elbow (622) and curved section (624) are orientated and configured to facilitate the second restraint portion (620) being positioned overtop of a fracture (660) in a meta-carpal (650) with which the splint (600) may be used.

The hinge (630) also provides a positioning function as it can lay against a side edge of a person's finger.

In-use, the curved section (624) extends around a person's knuckle which is substantially shaped by the second bone fragment (654).

The meta-carpal splint (660) is shaped and configured to enable the person's finger to extend from the flexed position shown in FIG. 28 to a substantively straight position (not shown in the Figures). This may be beneficial to facilitate use of the finger while the fracture (660) is healing. However, the first restraint portion (610) and the second restraint portion (620) are positioned on distal (either) sides of the fracture (660) to each other. Accordingly, the first restraint portion (610) and the second restraint portion (620) are able to orientate the first and second bone fragments (652, 654) in a position corresponding to a non-fractured bone. In addition, the first and second restraint portions (610, 620) facilitate holding the first and second bone fragments (652, 654) in the desired orientation. This is shown in FIG. 28.

Aspects of the present invention have been described by way of example only and it should be appreciated that modifications and additions may be made thereto without departing from the scope thereof as defined in the appended claims.

What is claimed is:

1. A splint for use in reducing a bone fracture in a body part that has a first surface and a second surface, wherein the bone fracture has produced at least a first bone fragment and a second bone fragment, wherein the splint includes:
a first restraint portion configured to apply pressure directly to the first surface and thereby apply pressure to the first bone fragment,
a second restraint portion configured to apply pressure directly to the second surface and thereby apply pressure to the second bone fragment,
wherein the orientation of the first restraint portion and the second restraint portion can be adjusted with respect to each other to thereby align the first bone fragment and the second bone fragment in a desired orientation that substantially corresponds to an orientation for the first bone fragment and the second bone fragment in a non-fractured bone, and
a connection assembly between the first restraint portion and the second restraint portion, and wherein the connection assembly can hold the first restraint portion and the second restraint portion substantially immovably with respect to each other to thereby hold the first bone fragment and the second bone fragment in the desired orientation.

2. The splint as claimed in claim 1, wherein the connection assembly includes a locking mechanism that includes at least one spacer element and a joint.

3. The splint as claimed claim 2, wherein the splint includes a locking mechanism to prevent movement about the joint.

4. The splint as claimed in claim 3, wherein the first restraint portion is substantially rigid.

5. The splint as claimed in claim 4, wherein the second restraint portion is substantially rigid.

6. The splint as claimed in claim 1, wherein the splint is a wrist splint.

7. The splint as claimed in claim 5, wherein the first restraint portion is configured to abut a volar surface of a person's forearm.

8. The splint as claimed in claim 7, wherein the second restraint portion is configured to abut a dorsal surface of a person's forearm and hand.

9. The splint as claimed in claim 1, including a third restraint portion moveably secured to the second restraint portion by a joint.

10. The splint as claimed in claim 9, including a locking mechanism to prevent movement about the joint between the third restraint portion and the second restraint portion.

11. The splint as claimed in claim 1, wherein the splint is an ankle splint.

12. The splint as claimed in claim 1, wherein at least one of the first restraint portion and the second restraint portion includes a coating layer on a surface which in use contacts a body part.

13. The splint as claimed in claim 12, wherein the coating layer is a foam material.

14. The splint as claimed in claim 12, wherein the coating layer is replaceable.

15. The splint as claimed in claim 1, including a length adjustment mechanism.

16. The splint as claimed in claim 15, wherein the length adjustment mechanism is a ratchet—type mechanism.

17. The splint as claimed in claim 1, wherein the splint is a meta-carpal splint.

18. The splint as claimed in claim 17, wherein the connection assembly is a hinge.

19. The splint as claimed in claim 18, wherein the hinge is resilient.

20. The splint as claimed in claim 18, including a portion to partially extend around a patient's knuckle in use.

21. The splint as claimed in claim 18, wherein the splint as formed as a one—piece, integral part.

22. The splint as claimed in claim 18, wherein the first restraint portion is configured to in use lay against an underside of a patient's finger and the second restraint portion is configured to lay against a top surface of a patient's finger such that the first and second restraint portions are on distal sides of a fracture in a patient's meta-carpal to each other.

23. The splint as claimed in claim 18, wherein the connection assembly is shaped and configured to align the splint in an operative position adjacent a patient's meta-carpal.

24. The splint as claimed in claim 18, wherein at least one of the first restraint portion and the second restraint portion is shaped to allow movement through a range of motion about a joint in the finger adjacent a fracture.

25. The splint as claimed in claim 24, wherein the range of motion is a substantially normal range of motion for the joint.
